(19) United States
(12) Patent Application Publication
(10) Pub. No.: US 2025/0034525 A1
(43) Pub. Date: Jan. 30, 2025

(54) CONSTRUCTION METHOD, DETECTION METHOD, AND APPLICATION OF IN VITRO TISSUE MODEL OF DIABETIC CARDIOMYOPATHY

(71) Applicant: HUBEI UNIVERSITY, Wuhan (CN)

(72) Inventors: Donghui ZHANG, Wuhan (CN); Lin CAI, Wuhan (CN); Yuxin ZHAO, Wuhan (CN); Qian LIU, Wuhan (CN)

(73) Assignee: HUBEI UNIVERSITY, Wuhan (CN)

(21) Appl. No.: 18/784,160

(22) Filed: Jul. 25, 2024

(30) Foreign Application Priority Data
Jul. 26, 2023 (CN) 202310928196.X

(51) Int. Cl. C12N 5/077

(52) U.S. Cl. CPC C12N 5/0657; C12N 2500/30; C12N 2500/38; C12N 2500/84; C12N 2501/734; C12N 2501/998; C12N 2501/999; C12N 2503/02; C12N 2503/04; C12N 2506/45; C12N 2513/00

(57) ABSTRACT

A construction method, a detection method, and application of an in vitro tissue model of diabetic cardiomyopathy. Bundles of human engineered heart tissue constructed from cardiomyocytes differentiated from induced human pluripotent stem cells are utilized to construct a disease model that simulates diabetic cardiomyopathy in which the myocardium evolves from diastolic dysfunction to systolic dysfunction upon stimulation. An in vitro tissue model of diabetic cardiomyopathy and a detection system based on altered contractile and electrical conduction properties for the same are constructed for the first time, and it is determined that a high concentration of PA is capable of inducing a change in the myocardium from unchanged contractility (impaired) to decreased contractility.

### CROSS-REFERENCE TO RELATED APPLICATIONS

This patent application claims the benefit and priority of Chinese Patent Application No. 202310928196.X filed with the China National Intellectual Property Administration on Jul. 26, 2023, entitled “A Construction Method, Detection Method and Application of In Vitro Tissue Model of Diabetic Cardiomyopathy”, the disclosure of which is incorporated by reference herein in its entirety as part of the present application.

### TECHNICAL FIELD

The present disclosure belongs to the technical field of cell engineering and specifically relates to construction method, detection method, and application of an in vitro tissue model of diabetic cardiomyopathy.

### BACKGROUND

Diabetes mellitus (DM), characterized by persistent hyperglycemia, has become a major threat to human health, with 537 million diabetic patients globally by 2022, and every 1 in 10 adults is suffering from diabetes. Diabetic cardiomyopathy (DCM) is the most common complication of diabetes and is a myocardial disease independent of cardiovascular-related factors such as hypertension, coronary artery heart disease, and valvular heart disease, its diagnostic criteria include left ventricular diastolic dysfunction, decreased systolic function, interstitial fibrosis and so on. Although the pathological process of myocardial structural and functional injuries occur in DCM, the decline in systolic function in the late stage is already very serious, and it is even more difficult to be treated than heart failure caused by ischemia alone, since it is a complex lesion caused by abnormalities in the body's metabolism, which is regulated by multiple factors and is insidious (it is easy to be ignored due to normal cardiac systolic function in the early stage). So far, there is still a lack of effective early diagnosis and treatment strategies for diabetic cardiomyopathy. How to effectively stimulate the DCM process, especially the progression from simple myocardial diastolic dysfunction to myocardial systolic dysfunction at the tissue level is key to solving the problems of early diagnosis and finding therapeutic drugs for DCM.

Currently, tissue models of DCM include traditional animal models and in vitro tissue models, in which high glucose/high-fat diet plus drugs are used to induce animal models such as transgenic mice and rats as diabetic models for the study of myocardial injury, but the species differences and the long period of the animal experiments are the important limiting factors. Studies on in vitro tissue modeling are limited by the characteristics of low regenerative capacity of cardiomyocytes and by the difficulty of obtaining cardiomyocytes of a human donor, which are currently underdeveloped. Induced human pluripotent stem cell (hPSC) is an ideal and popular seed cell, which has important potential for application in fields such as disease modeling, drug screening, and regenerative medicine therapy. By regulating Wnt (Wingless-Integrated) signaling, hPSC can be induced to differentiate into cardiomyocytes (hPSC-CM) orientated, and further combined with tissue engineering technology, hPSC-CM can be mixed with hydrogel to construct bundle shape, patch, or other human engineered heart tissue (hEHT, referred to as myocardial microtissues) that is suitable for detection, which not only can promote the maturation of hPSC-CM, but also can achieve disease characterization simulation and in vitro model construction by gene editing at the stem cell stage and changing the culture environment of hEHT. Currently, hPSC-CM-based diabetic cardiomyopathy models are mostly planar cells, and pathological phenotypes such as tissue fibrosis, and the existing tissue models fail to simulate the dynamic changes of myocardial contractile and electrical conduction properties at a tissue level.

### SUMMARY

An object of the present disclosure is to provide a construction method, a detection method, and application of an in vitro tissue model of diabetic cardiomyopathy, to provide more options for in vitro heart tissue models for studying the structural and functional injury of the heart induced by obesity, diabetes, etc., and to promote the application of cardiac micro-organ models constructed on the basis of human stem cell-derived cardiomyocytes in the cardiac disease-related fields.

The present disclosure provides a method for constructing an in vitro tissue model of diabetic cardiomyopathy, including: treating a human engineered heart tissue bundle with palmitic acid (PA) to obtain an in vitro tissue model of diabetic cardiomyopathy.

In some embodiments, a concentration of the palmitic acid for treating is 250-1000 μM; and a time for treating is 6-72 h.

In some embodiments, the method of constructing the human engineered heart tissue bundle includes, but is not limited to, the following steps:
(1) inducing differentiation of human pluripotent stem cells into human-derived cardiomyocytes, digesting the cardiomyocytes sequentially with collagenase and trypsin, centrifuging a resulting suspension of the cells, and re-suspending a resulting precipitate by centrifugation with Medium A to obtain a solution of the cells; wherein the Medium A includes a low-glucose DMEM (Dulbecco's Modified Eagle Medium) as a basal medium, and further includes fetal bovine serum (FBS), Penicillin-Streptomycin (PS), vitamin B12, and aminoacetic acid;
(2) mixing the solution of the cells with Thrombin, 2×Medium A, Matrigel, and Fibronectin to formulate a mixed system; and
(3) placing the mixed system in a cardiac bundle mold with a supporting frame, culturing and curing the mixed system to obtain a heart tissue bundle, and culturing the heart tissue bundle with an EHT medium to obtain the human engineered heart tissue bundle.

In some embodiments, the human-derived cardiomyocytes in step (1) have a purity of from 50% to 90%, preferably from 50% to 70%.

In some embodiments, the Medium A in step (1) includes low-glucose DMEM, 1%-20% fetal bovine serum, 1% Penicillin-Streptomycin, 1-4 μg/mL vitamin B12, and 0.5-5 mg/mL aminoacetic acid.

In some embodiments, the EHT medium in step (3) includes RPMI (Roswell Park Memorial Institute) 1640, 50×B27, 1% Penicillin-Streptomycin (m/m), 0.1-1 mg/mL ascorbic acid, 1-5 mg/mL aminoacetic acid, 0.1-2 μM 1-thioglycerol, 100×non-essential amino acids, and 100×sodiumpyruvate.

In some embodiments, 3D printing is utilized in step (3) to obtain the cardiac bundle mold with supporting frame.

The present disclosure also provides an in vitro tissue model of diabetic cardiomyopathy obtained using the above construction method.

In some embodiments of the in vitro tissue model, a concentration of the palmitic acid for treating is 250-1000 μM; and a time for treating is 6-72 h.

In some embodiments of the in vitro tissue model, the human engineered heart tissue bundle is constructed with the following steps:
(1) inducing differentiation of human pluripotent stem cells into human-derived cardiomyocytes, digesting the cardiomyocytes sequentially with collagenase and trypsin, centrifuging a resulting suspension of the cells, and re-suspending a resulting precipitate by centrifugation with Medium A to obtain a solution of the cells; wherein Medium A comprises a low-glucose DMEM as a basal medium, and further comprises fetal bovine serum, Penicillin-Streptomycin, vitamin B12, and aminoacetic acid;
(2) mixing the solution of the cells with Thrombin, Medium A, Matrigel, and Fibronectin to formulate a mixed system; and
(3) placing the mixed system in a cardiac bundle mold with a supporting frame, performing culturing and curing the mixed system to obtain a heart tissue bundle, and culturing the heart tissue bundle with an EHT medium to obtain the human engineered heart tissue bundle.

In some embodiments of the in vitro tissue model, the human-derived cardiomyocytes in step (1) have a cardiomyocyte purity of from 50% to 90%.

In some embodiments of the in vitro tissue model, the human-derived cardiomyocytes in step (1) have a cardiomyocyte purity of from 50% to 70%.

In some embodiments of the in vitro tissue model, the Medium A in step (1) comprises: low-glucose DMEM, 1%-20% fetal bovine serum, 1% Penicillin-Streptomycin, 1-4 μg/mL vitamin B12, and 0.5-5 mg/mL aminoacetic acid.

In some embodiments of the in vitro tissue model, the EHT medium in step (3) comprises: RPMI 1640, 50×B27, 1% Penicillin-Streptomycin, 0.1-1 mg/mL ascorbic acid, 1-5 mg/mL aminoacetic acid, 0.1-2 μM 1-thioglycerol, 100×non-essential amino acids, and 100×sodium pyruvate.

The present disclosure also provides a method for detecting the above-described in vitro tissue model of diabetic cardiomyopathy, wherein direct changes in the contractile function and electrical conduction function of the tissue are used as indicators of the detection.

The present disclosure also provides the use of the above-described human engineered heart tissue bundles or the above-described in vitro tissue model of diabetic cardiomyopathy in screening or developing a drug for cardiomyopathies.

The present disclosure also provides the use of an anti-hyperglycemic drug, Empagliflozin, obtained by screening using the above-described in vitro tissue model of diabetic cardiomyopathy in the preparation of a drug for treating diabetic cardiomyopathy.

In some embodiments, a concentration of Empagliflozin is 1 μM-100 μM; and more preferably, 5 M-22 μM.

Beneficial effects: The present disclosure provides an in vitro tissue model of human diabetic cardiomyopathy, which is constructed based on human cells, can make up for the defects that the planar cardiomyocyte model is unable to represent the overall tissue phenotype, and avoid the drawbacks caused by the species difference between animal models and humans. The model described in the present disclosure can simulate the real progression of diabetic cardiomyopathy, from the abnormal relaxation function at the tissue level in the heart to the decline of contractile function, by taking the contractility and electrical conduction function, which are the two most core functions of the heart as an important circulatory organ, as the output phenotype of the disease model.

In the present disclosure, bundles of human engineered heart tissue constructed from cardiomyocytes differentiated from induced human pluripotent stem cells are utilized to construct a disease model that simulates diabetic cardiomyopathy in which the myocardium evolves from diastolic dysfunction to systolic dysfunction upon stimulation. In the present disclosure, it is determined that high concentrations of palmitic acid have the most pronounced injury on cardiomyocytes with multiple factors' stimulation to the in vitro differentiated human-derived cardiomyocytes by a mechanical property testing platform, and further testing of the injury concentration determined that PA (palmitic acid) concentrations of 250-1000 μM and dynamic stimulation on hEHT produced a myocardial contractile force that ranged from unchanged (1 day) to decreased (3 days), and a decrease in the tissue's electrical conductivity, which was accompanied by an increase in the myocardial injury marker LDH (lactate dehydrogenase), as well as the development of a diabetic cardiomyopathy tissue model with myocardial fibrosis, myocardial sarcomere disorder, and mitochondrial injury. In the present disclosure, an in vitro tissue model of diabetic cardiomyopathy and detection system based on altered contractile and electrical conduction properties for the same are constructed for the first time, and it is determined that a high concentration of PA is capable of inducing a change in the myocardium from unchanged contractility (impaired) to decreased contractility. The model constructed in the present disclosure may be useful in screening therapeutic drugs for diabetic cardiomyopathy and lipotoxic cardiomyopathy.

In summary, the present disclosure provides more options for in vitro heart tissue models for studying the structural and functional injury of the heart induced by obesity, diabetes, etc., and promotes the application of cardiac micro-organ models constructed on the basis of human stem cell-derived cardiomyocytes in the cardiac disease-related fields, and has a wide range of prospects for research on the mechanism of the relevant diseases and drug screening, and thus has good practical application value.

### BRIEF DESCRIPTION OF THE DRAWINGS

The nature and mode of operation of the present disclosure will now be more fully described in the following detailed description taken with the accompanying drawing figures, in which:

FIG. 1A shows a picture of a polydimethylsiloxane mold for making human engineered heart tissue (hEHT) according to aspects of the present disclosure;

FIG. 1B is a schematic diagram of hEHT preparation using the mold according to aspects of the present disclosure;

FIG. 1C is a presentative image of the fabricated hEHT according to aspects of the present disclosure;

FIG. 1D is an image of a software interface displaying contractility data of hEHT according to aspects of the present disclosure;

FIG. 1E is a schematic diagram of hEHT contraction force analysis data according to aspects of the present disclosure;

FIG. 2A shows the results of stimulating human pluripotent stem cell-derived cardiomyocytes by Cell Counting Kit-8 (CCK8) for 24 hours according to aspects of the present disclosure;

FIG. 2B shows the results of stimulating human pluripotent stem cell-derived cardiomyocytes by CCK8 for 48 hours according to aspects of the present disclosure;

FIG. 3A shows the results by CCK8 of stimulating hPSC-CMs with different concentrations of PA stimulation for 24 hours, 48 hours, and 72 hours according to aspects of the present disclosure;

FIG. 3B shows the results of LDH detection in the culture supernatant according to aspects of the present disclosure;

FIG. 4A shows the results of the contraction force under different stretch conditions according to aspects of the present disclosure;

FIG. 4B shows the analysis of the diastolic duration and the percentage of the number of abnormal diastolic peaks that appeared under different stretch conditions according to aspects of the present disclosure;

FIG. 4C shows the analysis of the diastolic duration and the percentage of the number of abnormal diastolic peaks that appeared under different stretch conditions according to aspects of the present disclosure;

FIG. 4D shows the morphology of the tissue of hEHT by H&E and immunofluorescence assay and representative images of myocardial fibrosis according to aspects of the present disclosure;

FIG. 5A represents conduction velocity of tissues of hEHT by optical mapping after 6 hours' stimulation with 500 μM PA according to aspects of the present disclosure;

FIG. 5B represents contraction amplitude of tissues of hEHT by optical mapping after 6 hours' stimulation with 500 μM PA according to aspects of the present disclosure;

FIG. 5C represents APD (action potential duration) 80 of tissues of hEHT by optical mapping after 6 hours' stimulation with 500 μM PA according to aspects of the present disclosure;

FIG. 5D represents APD (action potential duration) 50 of tissues of hEHT by optical mapping after 6 hours' stimulation with 500 μM PA according to aspects of the present disclosure;

FIG. 6A shows the results of the contraction force under different stretch conditions according to aspects of the present disclosure;

FIG. 6B shows the analysis of the diastolic duration and the percentage of abnormal diastolic peak numbers that appeared under different stretch conditions according to aspects of the present disclosure;

FIG. 6C shows the analysis of the diastolic duration and the percentage of abnormal diastolic peak numbers that appeared under different stretch conditions according to aspects of the present disclosure;

FIG. 6D shows the representative images of the histological morphology and myocardial fibrosis of hEHT detected by H&E and immunofluorescence, respectively, according to aspects of the present disclosure;

FIG. 7A shows the effects of different concentrations of Empagliflozin (Em) on the survival rate of hPSC-CM induced by PA according to aspects of the present disclosure;

FIGS. 7B-7D show measuring the conduction velocity, amplitude and APD80 of the different groups by optical mapping after 24 hours' stimulation according to aspects of the present disclosure; and

FIG. 7E shows the percentage of the number of abnormal diastolic peaks of hEHT in different stimulation and dosing groups under stretch conditions according to aspects of the present disclosure.

### DETAILED DESCRIPTION OF THE EMBODIMENTS

An object of the present disclosure is to provide a construction method, a detection method, and application of an in vitro tissue model of diabetic cardiomyopathy, to provide more options for in vitro heart tissue models for studying the structural and functional injury of the heart induced by obesity, diabetes, etc., and to promote the application of cardiac micro-organ models constructed on the basis of human stem cell-derived cardiomyocytes in the cardiac disease-related fields.

The present disclosure provides an in vitro tissue model of human diabetic cardiomyopathy, which is constructed based on human cells, can make up for the defects that the planar cardiomyocyte model is unable to represent the overall tissue phenotype, and avoid the drawbacks caused by the species difference between animal models and humans. The model described in the present disclosure can simulate the real progression of diabetic cardiomyopathy, from the abnormal relaxation function at the tissue level in the heart to the decline of contractile function, by taking the contractility and electrical conduction function, which are the two most core functions of the heart as an important circulatory organ, as the output phenotype of the disease model.

In the present disclosure, bundles of human engineered heart tissue constructed from cardiomyocytes differentiated from induced human pluripotent stem cells are utilized to construct a disease model that simulates diabetic cardiomyopathy in which the myocardium evolves from diastolic dysfunction to systolic dysfunction upon stimulation. In the present disclosure, it is determined that high concentrations of palmitic acid have the most pronounced injury on cardiomyocytes with multiple factors' stimulation to the in vitro differentiated human-derived cardiomyocytes by a mechanical property testing platform, and further testing of the injury concentration determined that PA (palmitic acid) concentrations of 250-1000 μM and dynamic stimulation on hEHT produced a myocardial contractile force that ranged from unchanged (1 day) to decreased (3 days), and a decrease in the tissue's electrical conductivity, which was accompanied by an increase in the myocardial injury marker LDH, as well as the development of a diabetic cardiomyopathy tissue model with myocardial fibrosis, myocardial sarcomere disorder, and mitochondrial injury. In the present disclosure, an in vitro tissue model of diabetic cardiomyopathy and detection system based on altered contractile and electrical conduction properties for the same are constructed for the first time, and it is determined that a high concentration of PA is capable of inducing a change in the myocardium from unchanged contractility (impaired) to decreased contractility. The model constructed in the present disclosure may be useful in screening therapeutic drugs for diabetic cardiomyopathy and lipotoxic cardiomyopathy.

The present disclosure provides more options for in vitro heart tissue models for studying the structural and functional injury of the heart induced by obesity, diabetes, etc., and promotes the application of cardiac micro-organ models constructed on the basis of human stem cell-derived cardiomyocytes in the cardiac disease-related fields, and has a wide range of prospects for research on the mechanism of the relevant diseases and drug screening, and thus has good practical application value.

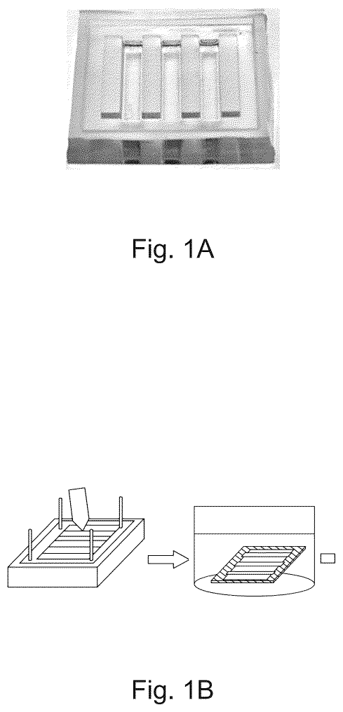

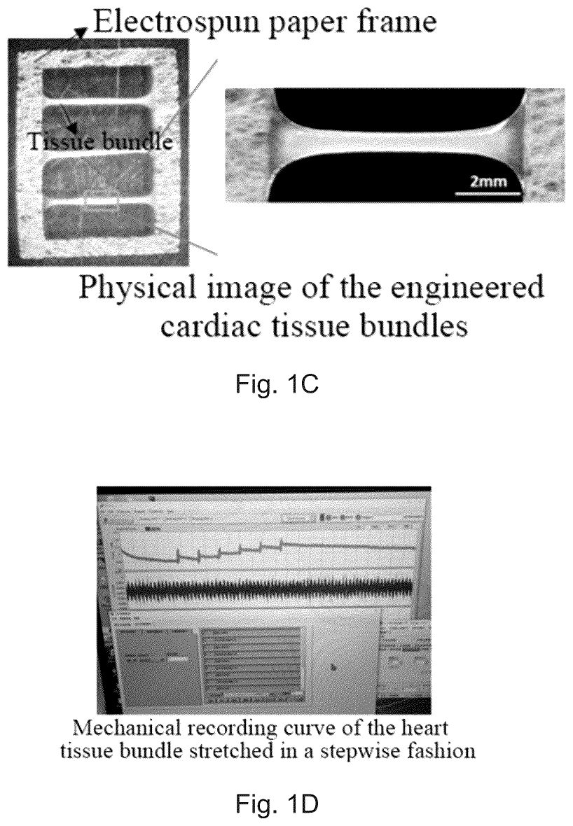

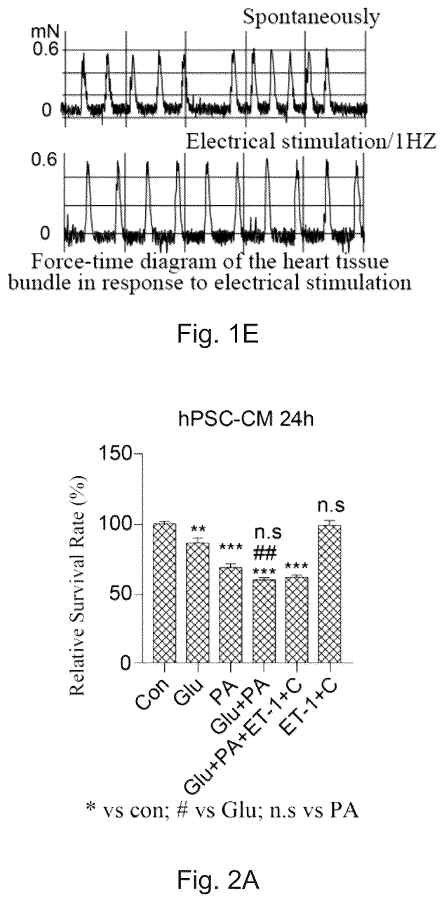

Referring now to the figures, FIG. 1A-FIG. 1E show the construction of engineered human heart tissue in the examples and the contraction force detection and analysis in Example 3 of the present disclosure, respectively. FIG. 1A is a mold for making hEHT. FIG. 1B is a schematic diagram of hEHT preparation using the mold of FIG. 1A. FIG. 1C is a presentive image of the fabricated hEHT. FIG. 1D is an image of a software interface displaying contractility data of hEHT. FIG. 1E is a schematic diagram of hEHT contraction force analysis data.

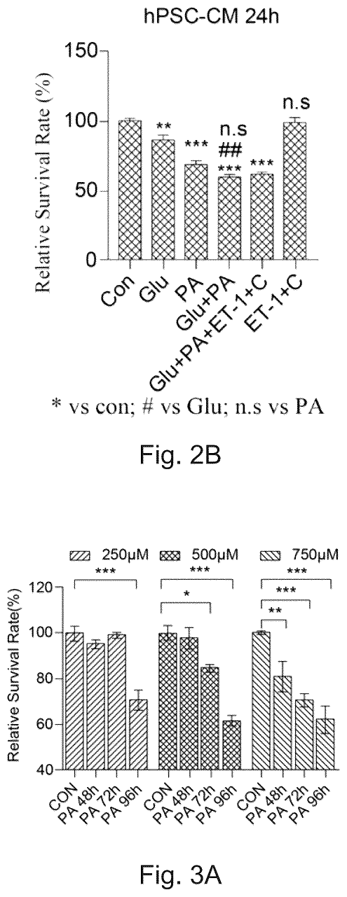

FIG. 2A-FIG. 2B show the results of confirming the effects of different metabolic factors on survival rate of the cardiomyocytes in the present disclosure. FIG. 2A shows the results of stimulating human pluripotent stem cell-derived cardiomyocytes by Cell Counting Kit-8 (CCK8) for 24 h with 30 mM Glucose, 250 μM PA, and 10 nM endothelin (ET-1)+1 mL glucocorticoid as single or combined factor(s), respectively, and the results indicate that the cardiomyocyte survival rate of the PA stimulation group was the lowest. FIG. 2B shows the results by CCK8 after stimulating for 48 h.

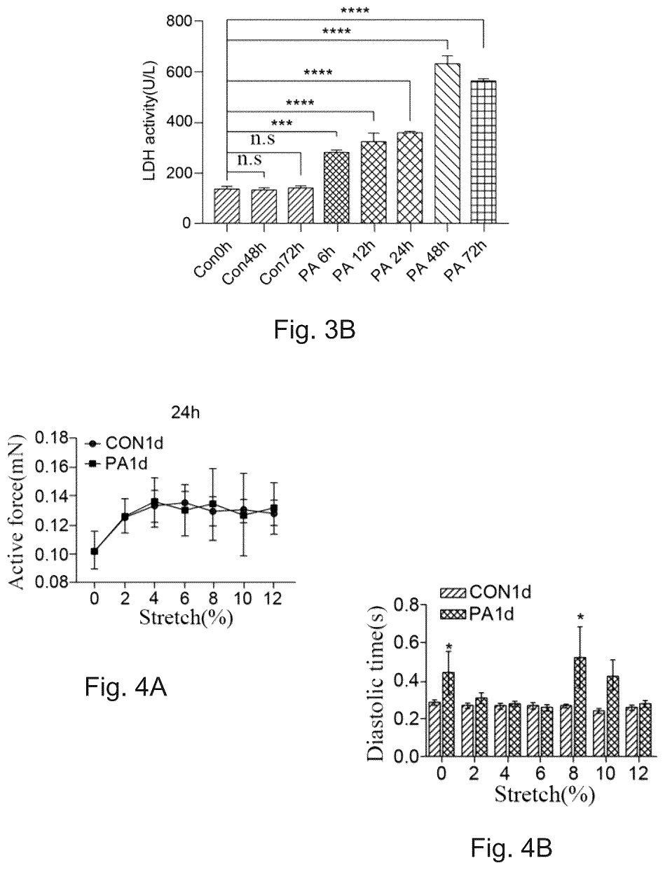

FIG. 3A-FIG. 3B show the results of confirming the effects of different concentrations of PA stimulation for different time points on the injury of human-derived cardiomyocytes in the present disclosure. FIG. 3A shows the results by CCK8 of stimulating hPSC-CMs with different concentrations of PA stimulation for 24 h, 48 h, and 72 h. FIG. 3B shows the results of LDH detection in the culture supernatant.

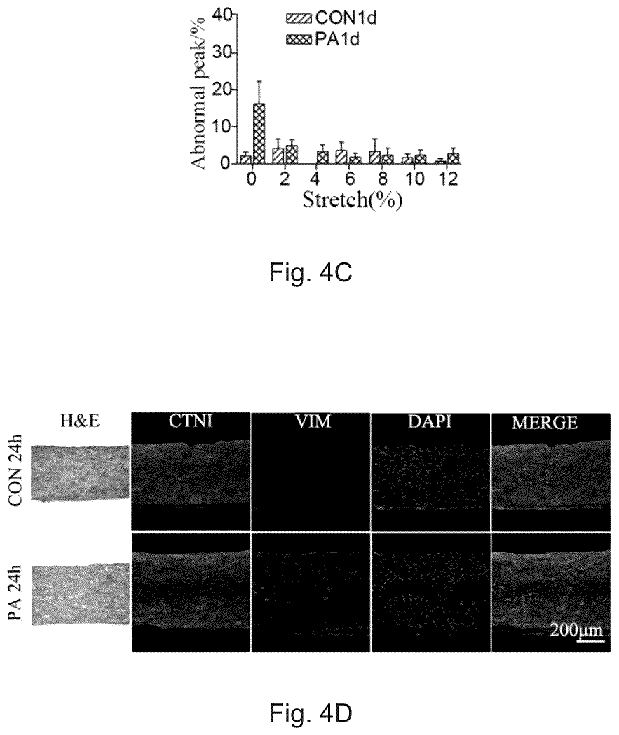

FIG. 4A-FIG. 4D show the results that only diastolic dysfunction, but no decreased contractility and obvious tissue fibrosis in hEHT stimulated with 500 μM PA for 24 h in the present disclosure. FIG. 4A shows the results of the contraction force under different stretch conditions. FIG. 4B and FIG. 4C show the analysis of the diastolic duration and the percentage of the number of abnormal diastolic peaks that appeared under different stretch conditions, respectively. FIG. 4D shows the morphology of the tissue of hEHT by H&E (hematoxylin and cosin) staining and immunofluorescence assay and representative images of myocardial fibrosis.

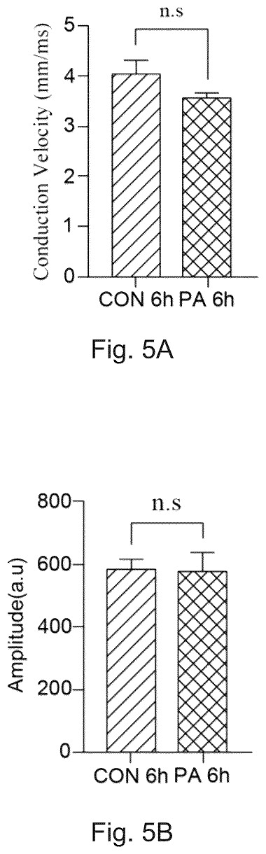

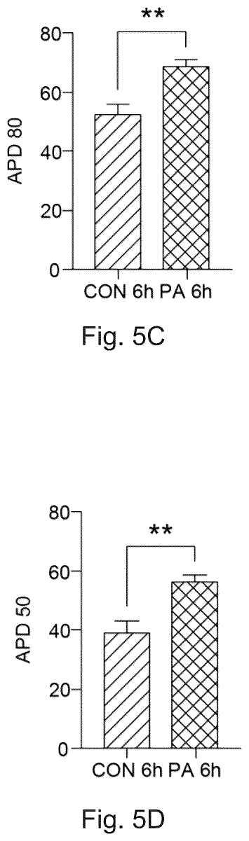

FIG. 5A-FIG. 5D show the results of measuring the electrical conductivity properties of tissues of hEHT by optical mapping after 6 hours' stimulation with 500 μM PA in the present disclosure. FIG. 5A represents conduction velocity. FIG. 5B represents contraction amplitude. FIG. 5C and FIG. 5D represent APD80 and APD50, respectively.

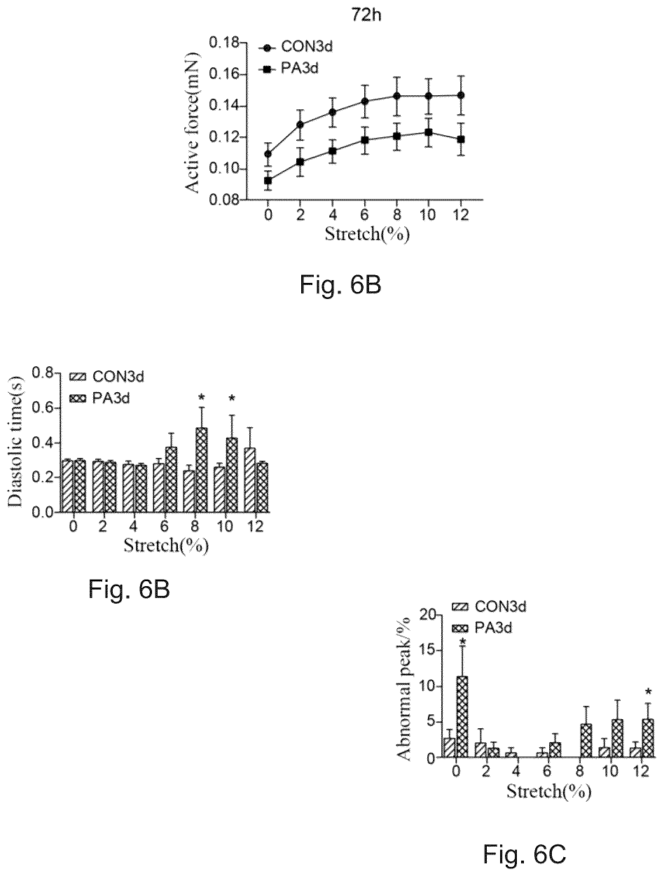

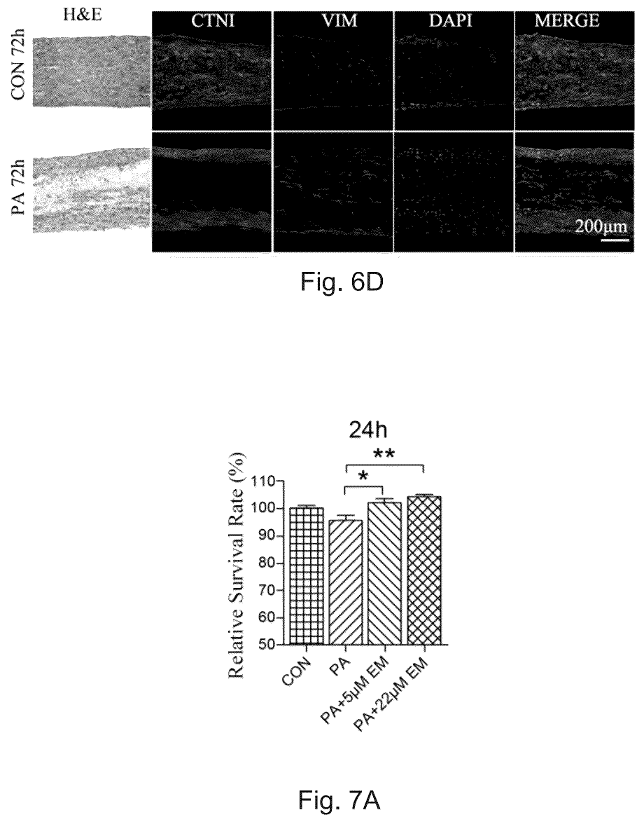

FIG. 6A-FIG. 6D show the results of decreased contractility and diastolic dysfunction, as well as obvious tissue fibrosis in hEHT stimulated with 500 μM PA for 72 h in the present disclosure. FIG. 6A shows the results of the contraction force under different stretch conditions. FIG. 6B and FIG. 6C show the analysis of the diastolic duration and the percentage of abnormal diastolic peak numbers that appeared under different stretch conditions, respectively. FIG. 6D shows the representative images of the histological morphology and myocardial fibrosis of hEHT detected by H&E and immunofluorescence, respectively.

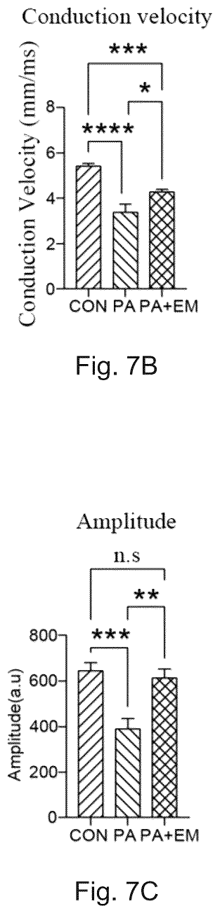

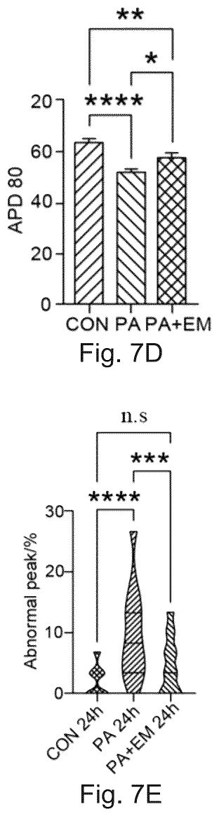

FIG. 7A-FIG. 7E show the results of validation of the anti-hyperglycemic drug, Empagliflozin, to improve the systolic and diastolic dysfunction of hEHTs induced by PA in the present disclosure. FIG. 7A shows the effects of different concentrations of Empagliflozin (Em) on the survival rate of hPSC-CM induced by PA. FIG. 7B-FIG. 7D show measuring the conduction velocity, amplitude and APD80 of the different groups by optical mapping after 24 hours' stimulation, respectively. FIG. 7E shows the percentage of the number of abnormal diastolic peaks of hEHT in different stimulation and dosing groups under stretch conditions.

In the present disclosure, a method for constructing an in vitro tissue model of diabetic cardiomyopathy is provided, including treating a human engineered heart tissue bundle with palmitic acid to obtain an in vitro tissue model of diabetic cardiomyopathy.

In the present disclosure, a concentration of palmitic acid is preferably 250-1000 μM; and a time for treating is preferably 6-72 h. In the present disclosure, anhydrous ethanol is preferably used to dissolve PA powder into a 250-mM concentrated stock solution; then prepare a PA stock solution: dissolve 1 g of BSA (bovine serum albumin) without fatty acid in 10 mL of DPBS (Dulbecco's Phosphate-Buffered Saline) without calcium and magnesium to prepare a 10% BSA solution, in which 100 μL of PA stock solution with a concentration of 250 mM is dissolved until it is clear and transparent in a 37° C. metal bath to prepare a 2.5 mM concentrated stock solution. In the present disclosure, cells or EHT medium are preferably used to dilute PA to a final concentration of 250-1000 μM.

In the present disclosure, the method of constructing a human engineered heart tissue bundle, preferably, includes the following steps:
(1) inducing differentiation of human pluripotent stem cells into human-derived cardiomyocytes, digesting the cardiomyocytes sequentially with collagenase and trypsin, centrifuging a resulting suspension of the cells, and re-suspending a resulting precipitate by centrifugation with Medium A to obtain a solution of the cells; wherein Medium A includes a low-glucose DMEM as a basal medium, and further includes fetal bovine serum, Penicillin-Streptomycin, vitamin B12, and aminoacetic acid;
(2) mixing the solution of the cells with Thrombin, Medium A, Matrigel, and Fibronectin to formulate a mixed system;
(3) placing the mixed system in a cardiac bundle mold with a supporting frame, performing culturing and curing to obtain a heart tissue bundle, and culturing the heart tissue bundle with an EHT medium to obtain the human engineered heart tissue bundle.

In the present disclosure, human pluripotent stem cells are induced to differentiate into human-derived cardiomyocytes, and collagenase and trypsin are sequentially used for digestion, the resulting cell suspension is centrifuged, and the centrifuged precipitate is resuspended by using Medium A to obtain a cell solution. The method for inducing differentiation is not particularly limited in the present disclosure, and hPSCs are preferably induced to differentiate into human-derived cardiomyocytes by using small chemical molecules, the example proves that the myocardial tissue bundles have better properties when prepared from human-derived cardiomyocytes with a cardiomyocyte purity of 50%-90%, more preferably, 50%-70%.

In the present disclosure, collagenase type I is preferably used to digest human-derived cardiomyocytes, and the digestion preferably includes digesting the human-derived cardiomyocytes at 37° C. for 45 minutes, collecting them into a centrifugal tube and standing for precipitation; after removing the supernatant, adding 1 mL of high-glucose DMEM to gently mix the cells well, and adding 9 mL of high-sugar DMEM continually to terminate the collagenase digestion.

In the present disclosure, trypsin is used for digestion after completing the collagenase digestion, and the digestion by trypsin preferably includes shaking the cells in a water bath at 37° C. for 3-5 minutes, and when there is no obvious cell cluster, trypsin digestion termination solution (high-glucose DMEM containing 50% FBS+DNase with a final concentration of 20 μg/mL) may be added, and the cell suspension is repeatedly blown until the DNA floc is completely digested. In the present disclosure, after the complete digestion, it is preferable to centrifuge the resulting cell suspension at 1000 rpm for 3 minutes, after removing the supernatant, resuspending the resulting precipitates with Medium A, and counting the number of the cells with a blood cell counting plate. The formula of the Medium A of the present disclosure preferably includes low-glucose DMEM, 1%-20% (m/m) fetal bovine serum, 1% (m/m) Penicillin-Streptomycin, 1-4 μg/mL vitamin B12 and 0.5-5 mg/mL aminoacetic acid; more preferably, it includes: low-glucose DMEM, 10% FBS, 1% PS, 2 μg/mL vitamin B12 and 1 mg/mL aminoacetic acid.

In the present disclosure, after the cell solution is prepared, it is mixed with Thrombin, 2×Medium A, Matrigel, and Fibronectin to prepare a mixed system. In the present disclosure, it is preferable to add 1.8 μL of Thrombin into 43.2 μL of the cell solution to form solution A, followed by mixing with solution B (18 μL 2×Medium A+9 μL Matrigel+18μL Fibronectin) in a volume ratio of 1:1 to form a mixed solution system of 90 μL.

After the mixed system is prepared, the mixed system is placed in a cardiac bundle mold with a supporting frame, performing culturing and curing to obtain a heart tissue bundle, and culturing the heart tissue bundle with an EHT medium to obtain the human engineered heart tissue bundle. In the present disclosure, the cardiac bundle mold with a supporting frame is preferably obtained by 3D printing, and the resulting mixed solution is quickly transferred into the bundle molding tool to form a bundle of 45 μL/bundle, which is cured in a 5% CO₂ incubator at 37° C. for 30 min, and the obtained bundle is transferred into a 12-well plate with a pair of tweezers.

In the present disclosure, the cells are preferably cultured on a shaker in an EHT medium. In order to prevent cell death, 10% FBS and 10 μM Y27632 are added on the first day of culture, and the spontaneous beating of EHT can be obviously observed on that day. The formula of the EHT medium of the present disclosure preferably includes RPMI 1640, 50×B27, 1% (m/m) Penicillin-Streptomycin, 0.1-1 mg/mL ascorbic acid, 1-5 mg/mL aminoacetic acid, 0.1-2 μM 1-thioglycerol, 100×non-essential amino acids and 100×sodium pyruvate. More preferably, RPMI 1640, 50×insulin-free B27, 1% (m/m) PS, 0.4 mg/mL ascorbic acid, 2 mg/mL aminoacetic acid, 0.45 μM 1-thioglycerol, 100×non-essential amino acids (including Glycine, L-Alanine, L-Asparagine, L-Aspartic acid, L-Glutamic Acid, L-Proline, L-Serine, 10 mM respectively) and 100×sodium pyruvate.

In the present disclosure, an in vitro tissue model of diabetic cardiomyopathy obtained by the above construction method is also provided. According to the disclosure, it is identified that a diabetic cardiomyopathy tissue model is generated with myocardial contractility that ranges from unchanged (1 day) to decreased (3 days), concomitant with the appearance of a myocardial fibrosis phenotype by dynamic stimulation of hEHT with 500 μM PA. Specifically, it is identified that PA stimulation ≤24 h (more preferably 6 h) can simulate early diabetic cardiomyopathy with myocardial diastolic dysfunction and partial impairment of electrical conductivity, but the contractility and fibrosis remain unchanged; when PA stimulation time increased to 72 h, which means advanced diabetic cardiomyopathy, in which the contractility and electrical conductivity decreased, obvious fibrosis appeared, sarcomere and mitochondria were injured, and LDH increased significantly.

In the present disclosure, a method for detecting the above in vitro tissue model of diabetic cardiomyopathy is also provided, in which the direct changes in the contractile function and electrical conduction function of the tissue are used as detection indicators.

In the present disclosure, a force transducer detection system is preferably used to directly detect the active contractile force and passive contraction force of EHT, and analyze mechanical contraction function indicators such as amplitude, ascending speed, descending speed, and APD50; and electrical conductivity speed, amplitude, ascending speed, descending speed, APD50, APD80 and other contractile function indicators of EHT are detected by optical mapping. In the present disclosure, it is also determined whether EHT is fibrotic by H&E staining and detects the distribution of fibroblasts inside EHT by immunofluorescence staining, thereby detecting the constructed in vitro tissue model.

In the present disclosure, the use of the above human engineered heart tissue bundle or the above in vitro tissue model of diabetic cardiomyopathy in screening or developing a drug for cardiomyopathies is also provided.

The in vitro tissue model obtained by the method of the present disclosure may be applied to carry out studies on pathogenesis and molecular targets of diabetic cardiomyopathy and lipotoxic cardiomyopathy; drug safety evaluation and research and development of new molecular drugs for diabetic cardiomyopathy and lipotoxic cardiomyopathy. The in vitro tissue model of the present disclosure may be used as an in vitro heart tissue model for screening drugs for diabetic cardiomyopathy, and based on this model, verifying the relieving effect of the anti-hyperglycemic drug Empagliflozin on diabetic cardiomyopathy.

In the present disclosure, the use of an anti-hyperglycemic drug, Empagliflozin, obtained by screening using the in vitro tissue model of diabetic cardiomyopathy in the preparation of a drug for treating diabetic cardiomyopathy is also provided.

In the present disclosure, it is also found that by using the in vitro tissue model of diabetic cardiomyopathy, an anti-hyperglycemic drug, Empagliflozin (the concentration of Empagliflozin is 1 μM-100 μM; more preferably, 5 μM-22 μM), has an excellent therapeutic effect on diabetic cardiomyopathy and may be used as a candidate drug for treating diabetic cardiomyopathy.

In order to further illustrate the present disclosure, the construction method, detection method, and the use of an in vitro tissue model of diabetic cardiomyopathy provided by the present disclosure will be described in detail with examples, which shall not be understood as limiting the protection scope of the present disclosure.

### Example 1

### Construction Method of Human Engineered Heart Tissue

HPSCs were differentiated into human cardiomyocytes using small chemical molecule induction (Cell Discov. 2022 8, 105), in which the myocardial tissue bundles prepared with a cardiomyocyte purity of 50%-70% exhibited better function. The cells were digested and replated on day 12-14 of cell differentiation. The detailed procedures are as follows:
1) A cardiac bundle mold and its paper supporting frame were prepared by a 3D printer: a fused deposition 3D printer (Shenzhen Aurora Technology CO., LTD) was used to print the positive mold of the EHT molding tool with an inner groove length of 12 mm and a width of 7 mm using polylactic acid as material; polydimethylsiloxane (PDMS) basement solution and the corresponding crosslinker were thoroughly mixed and poured into a glass jar containing the printed mold. The mixture was cured at 65° C. for 4 hours to obtain the muscle tissue strip molding template. After high-pressure steam sterilization, the surface hydrophilicity of the negative mold was increased by immersion in 0.2% F-127 for 2 hours. A laser engraving machine was used to cut nylon paper (Cerex) into paper frames with an outer width of 12.0 mm, an inner length of 7.0 mm. After high-pressure steam sterilization, the paper frames were placed in the negative mold as supporting paper frames for the myocardial tissue bundles. The results are shown in FIG. 1A-FIG. 1B.
2) The human-derived cardiomyocytes were digested with type I collagenase at 37° C. for 45 min, collected into a centrifugal tube, and left for precipitation;
3) After removing the supernatant by aspiration, 1 mL of high-glucose DMEM was added to gently mix the cells well, then 9 mL of high-glucose DMEM was added continually to terminate the collagenase digestion, the resulting solution was allowed for precipitation, after that, the supernatant was removed. The above procedures were repeated once again;
4) 1.5 mL of trypsin was added into the resulting precipitation in 3) above and the cells were blown off, which was placed into a water bath at 37° C. and shaken for 3-5 minutes to digest the cells. When there was no obvious cell mass, 3 mL of trypsin digestion termination solution (high-glucose DMEM containing 50% FBS+DNase I with a final concentration of 20 μg/mL was added, and the cell suspension was repeatedly blown until the DNA flocculent was completely digested;
5) The resulting cell suspension in 4) above was centrifuged at 1000 rpm for 3 minutes, and the supernatant was removed by aspiration, which was then resuspended with Medium A (formula: low-glucose DMEM+10% FBS+1% PS+2 μg/mL vitamin B12+1 mg/mL aminoacetic acid), and number of the cells was counted with a blood cell counting plate;
6) Preparation of heart tissue bundle: 1.8μL of Thrombin was added into 43.2 μL of Medium A containing cells to form solution A, followed by mixing with solution B (18 μL 2×Medium A+9 μL Matrigel+18 μL Fibronectin) in a volume ratio of 1:1 to form a mixed solution system of 90 μL. The resulting mixed solution was quickly transferred into a bundle molding mold to form bundles of 45 μL with each bundle containing 0.35-0.5 million cardiomyocytes, which were cured in a 5% CO₂ incubator at 37° C. for 30 min. The resulting bundle was transferred into a 12-well plate using tweezers.
7) The cells were cultured on a shaker with an EHT medium (RPMI 1640+50×insulin-free B27+1% PS+0.4 mg/mL ascorbic acid+2 mg/mL aminoacetic acid+0.45 μM 1-thioglycerol+100×non-essential amino acids+100×sodium pyruvate). In order to prevent cell death, 10% FBS and 10 μM Y27632 were added on the first day of culture, and the spontaneous beating of EHT could be obviously observed on that day. The image of EHT is shown in FIG. 1C.

### Example 2

### Preparation of High Concentration Palmitic Acid (PA) Solution and Drug Stimulation on EHTs

The effective concentration and the effect of PA depend on the dissolving method of PA. In the present disclosure, by means of using BSA to promote dissolution, the successful acquisition of a high-concentration PA solution was guaranteed.

1) Dissolving PA powder into a 250 mM concentrated stock solution with anhydrous ethanol;

2) Preparing a PA mother liquor: 1 g of BSA without fatty acid was dissolved in 10 mL of DPBS without calcium and magnesium to prepare a 10% BSA solution, in which 100μL of PA mother liquor with a concentration of 250 mM was dissolved until the resulting solution was clear and transparent in a metal bath at 37° C. to prepare a mother liquor with a concentration of 2.5 mM. In the present disclosure, cells or EHT medium were preferably used to dilute PA to a final concentration of 250-1000 μM.

3) Diluting PA with a final concentration of 250 μM, 500 μM, and 750 μM using cells or EHT medium to stimulate human-derived cardiomyocytes or EHTs. An equivalent amount of ethanol was dissolved in 10% BSA, which was diluted with medium finally as a control solution.

In the present disclosure, firstly, differentiated human cardiomyocytes were paved into planar monolayer cells, and then the cell survival rate was detected by adding 30 mM Glucose, 250 μM PA, 10 nM endothelin (ET-1) and 1 mL glucocorticoid to the medium as single or combined factor(s), respectively, it is identified that the most obvious diabetic stimulus factor for cardiomyocyte injury was PA (FIG. 2A-FIG. 2B). Furthermore, different concentrations of PA were used to stimulate cardiomyocytes, and the concentration and time-dependent effects were evaluated (FIG. 3A). The injury effect of PA was determined through myocardial injury marker LDH (FIG. 3B).

### Example 3

### Detection of Contractility in Human EHT Diabetic Cardiomyopathy Tissue Model

1) After EHT was constructed and cultured for 5-7 days (the medium was changed every other day), the EHTs had good structure and beat regularly.

2) EHT was stimulated by 500 μM PA, and for the control group, it contained the same concentration of BSA and ethanol. After 24 hours and 72 hours of stimulation, the supernatant and tissue samples were collected for functional detection.

3) A contractility detection system by mechanical assay: the mechanical characteristics of the bundle were detected by using a mechanical measurement platform, in which the bath, platinum electrode, tissue fixation device, and assembly form were self-designed, and other components such as sensors, amplifiers, digital-to-analog converters, data acquisition software, stepper motors, supporting components, temperature controllers and stimulators were all commercially available and assembled in-house after procurement.

4) Experimental steps for contractility detection by mechanical assay: the instrument was preheated for 30 minutes in advance, and the sensor was calibrated with precision weights by gravity. The Tyrode's Solution containing 1.8 mM CaCl₂ was put into a 37° C. water bath for preheating in advance, and the platform was set to 37° C. The bundle was dipped into the Tyrode's Solution, and both ends were fixed with specimen needles, wherein one of which was fixed on the sensor and the other was fixed on the bath. Data were collected after the microstructure was balanced for 2 min. Start the electrical stimulator, and stimulate the bundle electrically at a voltage of 10 V and frequencies of 1, 1.5, 2, 2.5, and 3 Hz. Start the stepper motor to control software to start the programmed stretching program, and stretch the bundle from the initial state (8 mm) by 12% (0.96 mm) of the initial length, stretching 2% (0.16 mm) each time, collecting data for 40 s in each stretching state, and collecting contraction force data under different stretching states. After stretching, end the collection and save the data. The collected data were analyzed by the customized program MATLAB software.

In the present disclosure, it was verified that stimulating human EHT with PA for 24 h did not affect the positive contractility (FIG. 4A), but did result in prolonged myocardial relaxation time (FIG. 4B) and abnormal relaxation peak (FIG. 4C). However, when the stimulation time was extended to 72 h, human EHTs exhibited a significant decrease in inotropic force (FIG. 6A) and more frequent prolongation of relaxation time and abnormal peaks (FIG. 6B-FIG. 6C). This result demonstrated in vitro that the heart developed from only diastolic dysfunction to both systolic and diastolic dysfunction in the late stage after diabetic stimulation of the myocardium. This experiment showed a functional change in heart tissue level that cannot be modeled by a single plane cardiomyocyte.

### Example 4

### Optical Mapping Detection of Human EHT Diabetic Cardiomyopathy Model

1) The electrode was made of 0.2 mm platinum wire, and the conductive function was tested using a sealed membrane nickel wire interface.

2) Fluo-4/RHOD2 was dissolved in DMSO (dimethyl sulfoxide) with a volume ratio of 1:1 to prepare a stock solution, which was then added into EHT medium at a ratio of 1:1,000 (working solution), incubated at 37° C. for 20 min, followed by washing with DPBS twice, then incubated with serum-free EHT medium at 37° C.

3) Preparation of medium for imaging: 10 μM Blebbistatin was added into the Tyrode's Solution or serum-free EHT medium in a volume ratio of 1:2,000 for imaging. The medium for imaging was added into a PDMS dish, the EHT was fixed on the dish with a specimen needle, put in a 41° C. temperature control chamber, and the electrode was put on one side of the EHT; an optical mapping camera was used for formal imaging, and the preview speed of 600 ms was recorded in 10 seconds, then recording was conducted in the cases where there was no electrical stimulation and 1 Hz electrical stimulation, respectively, in which the laser intensity was MAX: 12-16%.

The collected data were analyzed by optical mapping software.

Electrical conduction is one of the two most critical functions required for the heart to function as a whole. It was further identified in the present disclosure that the electric signal conduction velocity and the contraction amplitude of human EHT were not decreased when it was stimulated by PA for 6 h, but APD50 and APD80 were obviously increased (FIG. 5A-FIG. 5D), which comprehensively indicated that EHT had a prolonged relaxation time, suggesting that there might be diastolic dysfunction.

### Example 5

### Preparation and Staining of Pathological Specimens of Human Engineered Heart Tissue Bundles

For the morphological and histological observation after Bundle stimulation, the tissue was first treated by conventional paraffin embedding methods, and then the tissue was stained by basic staining and immunofluorescence staining after slicing. The specific steps were as follows:
(1) Cleaning the bundle twice using DPBS without calcium and magnesium ions.
(2) Fixing the bundle with 4% PFA (Paraformaldehyde) at room temperature for 15 min.
(3) Placing the fixed bundle in a paraffin tissue embedding frame and rinsing it with running water for 2 h.
(4) The paraffin-embedded bundle was subjected to gradient dehydrating and wax dipping using a Dewaxer (Hisure Scientific Co., Ltd.) with a total of 12 cylinders under the following conditions: 70% ethanol for 1 h, 80% ethanol for 1 h, 80% ethanol for 0.5 h, 90% ethanol for 0.5 h, 90% ethanol for 0.5 h, 100% ethanol for 0.5 h, xylene for 0.5 h, xylene for 0.5 h, paraffin at 63° C. for 5 min, paraffin at 63° C. for 5 min and paraffin at 63° C. for 5 min.
(5) After the dehydrated Bundle was placed at the bottom of the iron frame molding tool with an appropriate size, the plastic embedding frame was placed on the iron frame molding tool, and then placed on the freezing platform. After the paraffin was solidified, the iron frame mold was separated from the solid-paraffin-containing tissues.
(6) The bundle was cut into paraffin slides with a thickness of 6 μm using a rotary microtome.
(7) For the obtained paraffin slides, H&E staining was performed using the kit and conventional methods for fibrosis characterization of the bundle.
(8) The steps of immunofluorescence staining were as follows: 1) baking the slides at 65° C. for 1 h; 2) deparaffinizing with three xylene baths for 5 minutes each; 3) rehydration: 100% ethanol, 90% ethanol, 80% ethanol, 70% ethanol gradient rehydration, 5 min/cylinder; 4) antigen retrieval: 1×citrate buffer solution in a microwave until boiling, immerse tissue sections in the solution for 15 minutes, cooled to room temperature, and then washed with PBS for 5 min; 5) blocking: adding blocking solution containing 2% % Triton X-100 onto the tissue, and blocking at 37° C. for 1 h; 6) staining: removing excess blocking solution on the tissue, rinsing the tissue with PBS, adding primary antibody (1:200), and incubate overnight at 4° C. in the dark. The next day, the primary antibody on the tissue was removed and the tissue was washed with PBS for 5 minutes, then the corresponding secondary antibody was added dropwise and left at room temperature for 2 h in the dark, and the secondary antibody was washed off. After adding the anti-fluorescence quencher dropwise, the coverslip was covered, and the slide was mounted with nail polish.

The results of H&E staining and immunofluorescence staining of human EHT stimulated by PA for different times showed that there were no obvious pathological changes and changes in the level of fibrosis inside human EHT at 24 hours after PA stimulation (FIG. 4D), while obvious fibrosis was observed inside the heart tissue instead at 72 hours after PA stimulation (FIG. 6D). This finding pathologically validates the progression of fibrosis in different stages of diabetic cardiomyopathy.

### Example 6

Verification of the therapeutic effect of Empagliflozin on human diabetic cardiomyopathy.

Human EHT was constructed by the methods of Examples 1-4, and the effect was determined by adding Empagliflozin while stimulating with 500 μM of PA. In this case, Empagliflozin was dissolved in DMSO to formulate a concentration of 100 mM, followed by a gradient dilution of the drug with medium to the concentration used, which was used to stimulate hEHT along with PA in the relevant experiments.

In the present disclosure, it was verified that Empagliflozin at 5 μM and 22 μM could supplement the myocardial cell injury induced by PA (FIG. 7A). In human EHT, it was verified by optical mapping that Empagliflozin treatment could effectively recover the electric signal conduction velocity (FIG. 7B) and contraction amplitude (FIG. 7C) of human EHT from decline, and APD80 was improved (FIG. 7D), as well as reduced number of abnormal diastolic peaks (FIG. 7E). Direct detection of EHT contractility may also confirm the restoration of PA-induced decrease of EHT contractility by Empagliflozin. In this part, not only the excellent effect of the anti-hyperglycemic drug, Empagliflozin, on the treatment of diabetic cardiomyopathy was verified, but also the in vitro tissue model of diabetic cardiomyopathy established by the present disclosure could be used as a representative excellent model in the process of screening related drugs was verified.

While the foregoing embodiments have been described in detail, it is to be understood that these embodiments are merely illustrative of the disclosure and that the disclosure may be embodied in many other ways without departing from the spirit and scope of the disclosure. Accordingly, the foregoing embodiments are not to be construed as limiting the disclosure.

What is claimed is:

1. A method for constructing an in vitro tissue model of diabetic cardiomyopathy, the method comprising:
treating a human engineered heart tissue bundle with palmitic acid to obtain an in vitro tissue model of diabetic cardiomyopathy.

2. The method of claim 1, wherein a concentration of the palmitic acid for the treating is 250-1000 μM and a time for the treating is 6-72 h.

3. The method of claim 1, wherein the human engineered heart tissue bundle is constructed by the following steps:
(1) inducing differentiation of human pluripotent stem cells into human-derived cardiomyocytes, digesting the cardiomyocytes sequentially with collagenase and trypsin, centrifuging a resulting suspension of the cells, and re-suspending a resulting precipitate by centrifugation with Medium A to obtain a solution of the cells; wherein Medium A comprises a low-glucose DMEM as a basal medium, and further comprises fetal bovine serum, Penicillin-Streptomycin, vitamin B12, and aminoacetic acid;
(2) mixing the solution of the cells with Thrombin, Medium A, Matrigel, and Fibronectin to formulate a mixed system; and
(3) placing the mixed system in a cardiac bundle mold with a supporting frame, performing culturing and curing the mixed system to obtain a heart tissue bundle, and culturing the heart tissue bundle with an EHT medium to obtain the human engineered heart tissue bundle.

4. The method of claim 3, wherein the human-derived cardiomyocytes in step (1) have a cardiomyocyte purity of from 50% to 90%.

5. The method of claim 4, wherein the human-derived cardiomyocytes in step (1) have a cardiomyocyte purity of from 50% to 70%.

6. The method of claim 3, wherein the Medium A in step (1) comprises: low-glucose DMEM, 1%-20% fetal bovine serum, 1% Penicillin-Streptomycin, 1-4 μg/mL vitamin B12, and 0.5-5 mg/mL aminoacetic acid.

7. The method of claim 3, wherein the EHT medium in step (3) comprises: RPMI 1640, 50×B27, 1% Penicillin-Streptomycin, 0.1-1 mg/mL ascorbic acid, 1-5 mg/mL aminoacetic acid, 0.1-2 μM 1-thioglycerol, 100×non-essential amino acids, and 100×sodium pyruvate.

8. An in vitro tissue model of diabetic cardiomyopathy obtained using the method of claim 1.

9. The in vitro tissue model of diabetic cardiomyopathy of claim 8, wherein a concentration of the palmitic acid for the treating is 250-1000 μM and a time for the treating is 6-72 h.

10. The in vitro tissue model of diabetic cardiomyopathy of claim 8, wherein the human engineered heart tissue bundle is constructed by the following steps:
(1) inducing differentiation of human pluripotent stem cells into human-derived cardiomyocytes, digesting the cardiomyocytes sequentially with collagenase and trypsin, centrifuging a resulting suspension of the cells, and re-suspending a resulting precipitate by centrifugation with Medium A to obtain a solution of the cells; wherein Medium A comprises a low-glucose DMEM as a basal medium, and further comprises fetal bovine serum, Penicillin-Streptomycin, vitamin B12, and aminoacetic acid;
(2) mixing the solution of the cells with Thrombin, Medium A, Matrigel, and Fibronectin to formulate a mixed system; and
(3) placing the mixed system in a cardiac bundle mold with a supporting frame, performing culturing and curing the mixed system to obtain a heart tissue bundle, and culturing the heart tissue bundle with an EHT medium to obtain the human engineered heart tissue bundle.

11. The in vitro tissue model of diabetic cardiomyopathy of claim 10, wherein the human-derived cardiomyocytes in step (1) have a cardiomyocyte purity of from 50% to 90%.

12. The in vitro tissue model of diabetic cardiomyopathy of claim 11, wherein the human-derived cardiomyocytes in step (1) have a cardiomyocyte purity of from 50% to 70%.

13. The in vitro tissue model of diabetic cardiomyopathy of claim 10, wherein the Medium A in step (1) comprises: low-glucose DMEM, 1%-20% fetal bovine serum, 1% Penicillin-Streptomycin, 1-4 μg/mL vitamin B12, and 0.5-5 mg/mL aminoacetic acid.

14. The in vitro tissue model of diabetic cardiomyopathy of claim 10, wherein the EHT medium in step (3) comprises: RPMI 1640, 50×B27, 1% Penicillin-Streptomycin, 0.1-1 mg/mL ascorbic acid, 1-5 mg/mL aminoacetic acid, 0.1-2 μM 1-thioglycerol, 100×non-essential amino acids, and 100×sodium pyruvate.

15. A method for detecting the in vitro tissue model of diabetic cardiomyopathy of claim 8, wherein direct changes in the contractile function and electrical conduction function of the tissue are used as indicators for detection.

16. A method of screening or developing a drug for cardiomyopathies, comprising applying the in vitro tissue model of diabetic cardiomyopathy of claim 8.

17. A method of treating diabetic cardiomyopathy, comprising screening for Empagliflozin using the in vitro tissue model of diabetic cardiomyopathy of claim 8 and administering Empagliflozin to a subject in need thereof.
